(19) United States
(12) Patent Application Publication
(10) Pub. No.: US 2023/0233320 A1
(43) Pub. Date: Jul. 27, 2023

(54) TUBULAR INSTRUMENT WITH SELF-EXPANDING WIRE STRUCTURE

(71) Applicant: EPFLEX FEINWERKTECHNIK GMBH, Dettingen/Erms (DE)

(72) Inventor: Bernhard Uihlein, Dettingen (DE)

(73) Assignee: Cephea Valve Technologies, Inc., Abbott Park, IL (US)

(21) Appl. No.: 18/001,337

(22) PCT Filed: Aug. 3, 2021
(86) PCT No.: PCT/EP2021/071646

§ 371 Date: Dec. 9, 2022

(30) Foreign Application Priority Data
Aug. 4, 2020 (DE) 10 2020 209 823.9

(51) Int. Cl. A61F 2/24

(52) U.S. Cl. CPC A61F 2/2439; A61F 2/2418; A61F 2002/9511

(57) ABSTRACT

A tubular instrument includes a tubular assembly having first and second handling tubes extending from a proximal operating area to a distal functional area, a radially self-expanding wire structure, and a holding device. The first and second handling tubes are rotatable relative to each other. A control interface is provided at the proximal operating area for the relative rotational motion actuation of the first and second handling tubes. The holding device includes a first connecting unit and a second connecting unit. The first connecting unit includes a first filament connecting unit having a first connecting filament fixed to a first structural connection interface of the wire structure and to a tube connection interface of the first handling tube and extends therebetween with a radial directional component, and which winds onto or unwinds from the first handling tube upon rotation of the first and second handling tubes.

The invention relates to a tubular instrument comprising a tubular assembly having first and second handling tubes extending from a proximal operating area to a distal functional area, a radially self-expanding wire structure, and a holding device configured to hold the radially self-expanding wire structure at the distal functional area of the tubular assembly.

In some embodiments, the tubular assembly has only these two handling tubes or one or more further handling tubes, i.e., the tubular assembly comprises any number of two or more handling tubes. The handling tubes may be arranged with collapsing longitudinal nested axes, i.e., coaxially, or with parallel offset longitudinal axes lying one inside the other. Alternatively, they may be arranged with parallel longitudinal axes lying side by side. The handling tubes may be hollow tubes, i.e., actual tubes, or alternatively solid tubes, i.e., rods with a solid non-hollow cross-section, and in the case of a nested tube arrangement, at least the outermost tube is a hollow tube.

In particular, the tubular instrument may be a medical catheter instrument, e.g., one used for minimally invasive implantation of artificial heart valves by means of the so-called TAVI (transcatheter aortic valve implantation) technique and of similar medical implants with a radially self-expanding wire structure, such as stents and the like. Alternatively, the tubular instrument is also suitable for other medical and non-medical uses, e.g., for inserting plugs into conduits of water supply systems, pipeline systems and other systems designed to carry fluids. The wire structure can have an arbitrary, preferably wire mesh-like shape, e.g. a cylindrical shape, such as is suitable for applications in artificial heart valves and vascular stents. In other applications, the wire structure may have a different shape adapted to its intended use. In this respect, it is only essential that it has a radially self-expanding shape and that it is a structure made of elastic wire material that unfolds from a radially compressed configuration into a radially expanded configuration by virtue of inherent material properties or preload. Radial direction is therefore understood in the present context as a direction perpendicular to a longitudinal direction of the wire structure and thus transverse to the longitudinal direction of the tubular assembly at the distal end portion of which it is held. For this purpose, the wires can be made, for example, of a metallic alloy material with shape memory or another elastic metal or plastic material. The wire structure may be cut from a tube material by laser cutting, for example, as is common for stents. In an alternative manufacturing variant, it may be composed of several individually prefabricated structural parts and/or wire pieces.

The wire structure may be held by the holding device on the distal functional area of the tubular assembly in a detachable or non-detachable manner, i.e., not non-destructively detachable. For applications in which the wire structure should remain at a positioned location of use, e.g., for a heart valve or a vascular stent, a detachable embodiment is selected; for applications in which the wire structure serves only as a transport medium or carrier, to store another object or another substance at the respective location of use, a non-detachable embodiment can also be selected, since in this case the wire structure is removed from the location of use together with the tubular assembly.

For the above-mentioned applications in medical technology, tubular instruments of the type mentioned above and other types are commonly used in various embodiments. For example, the laid-open publication WO 2010/096176 A1 discloses a tubular instrument for implanting a prosthetic valve comprising a radially self-expanding stent frame having cell openings, and a crimping tool having a radially self-expanding wire structure, wherein the wire structure includes a plurality of circumferentially distributed wire prongs.

A well-known problem with tubular instruments of the type mentioned above and other types with radially self-expanding wire structure, such as those for implanting stent-based heart valves and vascular stents, i.e., vasodilating stents, is that precise placement of the wire structure at a desired location of use is often relatively difficult, and the renewed positioning, i.e., the repositioning, of the wire structure once it is set in place is generally not possible, or at least not without difficulty. This is because after the radially self-expanding wire structure is brought to the location of use, radial expansion of the wire structure is permitted to cause it to snag or otherwise bond to surrounding material, e.g., a heart valve or vascular stent bonds to surrounding body tissue. Axial displacement of the wire structure is then virtually impossible and could also lead to undesirable damage to the surrounding material in question.

This problem is addressed, for example, in the laid-open publication WO 2010/057262 A1. One type of atrioventricular prosthetic heart valve disclosed therein and, for example, also in the First Publication of the Patent Application WO 2007/100410 A2 is composed of two prosthetic components to be implanted sequentially, a radially self-expanding receiver component implanted first and a radially self-expanding valve component implanted subsequently, which is placed in the previously implanted receiver component and attached to it and/or surrounding body tissue.

The laid-open publication WO 2008/100600 A1 discloses a platform type for an implantable prosthetic heart valve in which an already implanted prosthetic valve serves as a platform for a new prosthetic valve to be implanted.

The laid-open publication WO 2007/051620 A1 discloses a device for transvascular replacement of diseased heart valves comprising a self-expanding flexible wire mesh, in the center of which is a prosthetic valve body and which, when released from a feed catheter tube, turns over outwardly in a fungiform into pockets of a diseased heart valve.

This everting configuration of the wire structure is designed to allow the wire mesh, along with the prosthetic valve body, to be pulled back into the catheter tube by everting in the event of a malposition or malfunction of the valve prosthesis body. This is accomplished by using guide wires to pull the wire mesh into the catheter tube at three or more distal holding punctures, which causes a fungiform proximal bead of the wire mesh to fold back, stretching the wire mesh and dislodging it from its anchorage in the cardiac valvular pockets.

The laid-open publication US 2002/0143387 A1 deals with the repositioning or removal of a vascular stent, for which purpose it is provided with a pull ring at least one of its ends. The pull ring may be gripped by a gripper element disposed at the distal end of a stent removal device having a control interface at the proximal end for manual operation of the gripper element. By pulling axially on the pull ring gripped by the gripper element, the stent contracts radially in this region, after which it can be retracted axially into a receiver tube or catheter tube.

The invention is based on the technical problem of providing a tubular instrument of the type mentioned at the onset, which offers advantages over the prior art explained above, in particular with regard to the precise positioning or repositioning of the self-expanding wire structure as required.

The invention solves this problem by providing a tubular instrument comprising the features of claim 1. Advantageous further developments of the invention, which contribute to the solution of this and further problems are indicated in the subclaims, the contents of which, including all combinations of features resulting from the claim references, are hereby rendered the contents of the description in their entirety, by means of reference.

In the tubular instrument according to the invention, the first and second handling tubes are arranged so that they can rotate relative to one another, whereby the term ‘relative rotational motion’ is understood to mean that only the first handling tube rotates about its longitudinal axis relative to the second handling tube, or only the second handling tube rotates about its longitudinal axis relative to the first handling tube, or both handling tubes rotate about their respective longitudinal axis relative to one another. The latter is preferred in many cases. A control interface is provided at the proximal operating area for the relative rotational motion actuation of the first and second handling tubes, referring to the fact that the two handling tubes can be actuated in such a way that they execute the relative rotational motion defined above, for which purpose preferably both handling tubes are actively rotated or, alternatively, only one or only the other handling tube is actively rotated. In this context, active rotational motion actuation of both tubes, i.e. the first and the second handling tube, in opposite directions is particularly favorable for some applications.

The holding device comprises a first connecting unit connecting the wire structure to the first handling tube, and a second connecting unit connecting the wire structure to the second handling tube. Depending on the requirements, these connections are realized in a detachable or non-detachable way, i.e., not non-destructively detachable. For applications in which the wire structure should remain at a positioned location of use, e.g., for a heart valve or a vascular stent, a detachable embodiment is selected; for applications in which the wire structure serves as a transport medium or carrier body, to store another object or another substance at the respective location of use, a non-detachable embodiment can also be selected, since in this case the wire structure is removed from the location of use together with the tubular assembly. The first connecting unit includes a first filament connecting unit comprising at least one first connecting filament which is fixed, on the one hand, to a first structural connection interface of the wire structure and, on the other hand, to a tube connection interface of the first handling tube and extends therebetween with a radial directional component, and which winds onto or unwinds from the first handling tube upon relative rotation of the first and second handling tubes in accordance with the direction of rotation.

Thus, in the case of the tubular instrument according to the invention, the user can cause a relative rotation of the first and second handling tubes, i.e., a relative rotation of these two tubes with respect to each other, around the respective longitudinal tube axis via the proximal control interface manually or by means of a motorized drive or other automated operating unit.

For this purpose, only the first or only the second or both handling tubes are actively rotated, depending on the system design and requirements. Optionally, the tubular assembly comprises one or more further handling tubes arranged for relative rotational movement with respect to one or more of the other handling tubes in the sense defined above. When the handling tubes are arranged coaxially, their longitudinal axes coincide; otherwise, they are arranged with their longitudinal axes offset in parallel, nested or side by side, whereby the mixed case is also possible, in which at least one tube lies inside another (nested) and at least one tube lies next to another. For the handling tubes, an embodiment of hollow material, e.g., hollow tube or hollow rod, or of solid material, e.g., solid tube or solid rod, is selected in each case in a desired and suitable manner.

The first connecting unit with the first filament connecting unit is used to connect the wire structure to the first handling tube, wherein the filament connecting unit includes one or multiple first connecting filament, each of which is fixed to the wire structure on the one hand and to the first handling tube on the other hand. The fixing to the wire structure takes place specifically at the first structural connection interface of the wire structure designed for this purpose, i.e., the first structural connection interface is to be understood in the present context as the totality of those points or areas of the wire structure to which a respective first connecting filament is fixed. Analogously, the coupling of the first connecting filament(s) to the first handling tube is realized in that the first connecting filament(s) is/are fixed to said tube connection interface of the first handling tube, i.e., the tube connection interface is to be understood in the present context as the entirety of the points or areas of the first handling tube to which a respective first connecting filament is fixed. Any conventional material suitable for this purpose can be used for the first connecting filaments, e.g., an elastic, preferably highly elastic, and highly stable thread, strand or yarn material.

The optional detachability of the connection between the wire structure and the first handling tube can be implemented as required and in accordance with the application in such a way that the connection of the first connecting filament(s) to the wire structure is designed to be detachable and/or the connection of the first connecting filament(s) to the first handling tube is designed to be detachable. For the respective detachable connection at the relevant connection point(s), any conventional implementations can be used, e.g., node connections that are detached under the effect of tensile force, or node connections that are detached when the effect of tensile force is reduced, connection nodes that can be detached by suitable tools, detachable adhesive connections and detachable interlocking connections.

When the wire structure is detachably connected by means of the holding device to both the first and second handling tubes respectively, it can be brought or maneuvered by a user to a desired location of use by appropriate automated or manual handling of the handling tubes and then remain in said place, for which purpose the connection to the handling tubes is detached so that the handling tubes can subsequently be repositioned or removed. Following the above method, for example, in the case of medical applications of the tubular instrument in the form of a corresponding catheter instrument, an artificial heart valve or a vasodilator stent body, e.g., vascular stent, can be placed at a desired location in the body of a patient.

The wire structure typically remains at the desired location of use in that, thanks to its radially self-expanding characteristics, it is allowed to automatically expand from a compressed feed configuration to an expanded use configuration after the wire structure has been brought to its location of use in its compressed configuration, e.g., by moving it axially out of a feed tube in which it is received in its compressed configuration. This radial expansion allows the wire structure to bond with radially surrounding material, e.g., a body tissue material in the case of medical catheter applications, or any other surrounding material in the case of non-medical applications, e.g., in water conduit systems and other piping systems carrying fluids.

In the tubular instrument according to the invention, the special configuration of the first connecting unit enables the wire structure to be repositioned in a very advantageous manner, if necessary. For this purpose, the user manually or automatically initiates a relative rotational motion of the two handling tubes against each other, as explained above. In particular, it can be convenient for many applications to actively rotate both handling tubes in opposite directions, i.e., one tube clockwise and the other counterclockwise. The relative rotation of the two handling tubes causes the first connecting filament(s) to wind onto the first handling tube if the direction of rotation of the two tubes is appropriate. Since the respective first connecting filament extends between the wire structure and the first handling tube with a radial directional component, its winding onto the first handling tube has the effect of shortening it radially in the process, as a result of which it pulls the wire structure radially inwards at the relevant first structural connection interface.

As a result, even if the wire structure has already bonded to radially surrounding material as a result of the radial expansion, it can be freed from this bond with the radially surrounding material again without this leading to unwanted damage to this surrounding material. The wire structure can then be moved axially for repositioning without this being significantly impeded by adjacent material or leading to unwanted damage to said material. This method also allows the wire structure to be moved completely axially and thus removed from its previous positioning location, if required, without any problems and without affecting the radially surrounding material or being obstructed by said material. By means of a relative rotation of the two handling tubes in the opposite direction to the winding-up relative rotation, the previously wound-up first connecting filament or filaments can be unwound again from the first handling tube so that they release the automatic radial expansion of the wire structure again, e.g., in order to reposition the wire structure on radially surrounding material. Once the wire structure has been accurately positioned at a desired location, it can remain there, and the handling tubes can be pulled off or removed after the connections of the wire structure to the handling tubes have been released. Alternatively, if it serves only as a transport medium, it can be retracted radially again after the transported object or medium has been positioned and removed together with the handling tubes.

The winding or unwinding of the first connecting filament(s) onto or from the first handling tube is effected by the fact that, due to the relative rotation of the first and second handling tubes, the tube connection interface of the first handling tube rotates with respect to the wire structure, including its first structural connection interface, that is, changes its rotational position with respect to that of the first structural connection interface of the wire structure, in that the tube connection interface of the first handling tube rotates in a rotationally fixed manner with the first handling tube as a whole or maintains its rotational position, while the wire structure, due to its additional connection to the second handling tube, does not follow an active rotation of the first handling tube relative to the second handling tube at all or at any rate not completely or at least partially follows an active rotation of the second handling tube, including its first structural connection interface at which the associated end of the first connecting filament or filaments is located.

In a further embodiment of the invention, the first filament connecting unit has a plurality of first connecting filaments, each of which is fixed on the one hand to the first structural connection interface of the wire structure and on the other hand to the tube connection interface of the first handling tube and extends therebetween with a radial directional component and winds onto or unwinds from the first handling tube during a relative rotation of the first and second handling tubes depending on the direction of rotation, that is, depending on the direction of relative rotation or change in rotational position of the two handling tubes with respect to each other.

This measure favors a uniform connection of the wire structure via its first structural connection interface to the first handling tube or its tube connection interface via the multiple connecting filaments in this case. Optionally, the wire structure or its first structural connection interface and/or the first handling tube may have a number of connecting points corresponding to the number of first connecting filament, to each of which one of the first connecting filaments may be attached.

Since each of these connecting filaments winds onto the first handling tube during corresponding relative rotation of the first and second handling tubes, this measure also advantageously supports uniform application of the radial retraction force provided by the first filament connecting unit during winding of the connecting filaments to the wire structure at a plurality of interspaced points in the region of the first structural connection interface thereof. In alternative embodiments, the first filament connecting unit comprises only a single connecting filament, which may be sufficient for certain applications.

In one embodiment of the invention, the first structural connection interface has a plurality of first filament attachment points distributed in a circumferential direction of the wire structure and to which the first connecting filaments are attached. For example, the first filament attachment points may be distributed, in particular, over an annular region of the wire structure. Correspondingly, the first connecting filaments can be fixed analogously, namely distributed in a circumferential direction, at the tube connection interface of the first handling tube. Depending on the need and application, one or more first connecting filaments can be fixed at the respective filament attachment point.

Via the first filament connecting unit configured as described above, a circumferentially distributed and thus uniform connection of the wire structure to the first handling tube and a correspondingly circumferentially relatively uniform action of the filament connecting unit for radially contracting the wire structure at least in the region of its first structure connection interface is ensured in a very advantageous manner. In this context, the first connecting filaments can be arranged in particular at equal distances from one another in the circumferential direction or, depending on requirements and the application, e.g., depending on the specific shape of the wire structure, in particular in the region of its first structural connection interface, alternatively in a distribution with different distances in the circumferential direction. In alternative implementations, the plurality of first connecting filaments are fixed at a common filament attachment point of the first structure connection interface of the wire structure, while they may be circumferentially distributed, for example, at the tube connection interface of the first handling tube.

In a further embodiment of the invention, the first and second handling tubes are arranged for relative movement with respect to each other in the axial direction parallel to the longitudinal axis of the tube. This enables an axial evasive movement between the connection of the wire structure to the tube connection interface of the first handling tube on the one hand and the connection of the wire structure to the second handling tube on the other hand, which is convenient for many applications. Given the above, the fact that the radial expansion of the wire structure can be accompanied by an axial shortening of said wire structure, and in the same way the radial contraction of the wire structure can be accompanied by an axial lengthening of said wire structure, can be easily dealt with. The relative axial mobility of the two handling tubes can easily absorb or compensate for this axial change in length of the wire structure during radial expansion or contraction. In alternative embodiments, the first and second handling tubes are rigidly coupled to each other in the axial direction, which may be sufficient for some applications.

In a further embodiment of the invention, the tube connection interface of the first handling tube has a filament holding element which is arranged on the first handling tube in a rotationally fixed and axially movable manner and on which the at least one first connecting filament is fixed. This provides an advantageous embodiment for securing the first connecting filament(s) to the tube connection interface of the first handling tube. Due to the rotationally fixed coupling, the filament holding element follows the first handling tube in its rotational position, thereby ensuring the desired winding or unwinding of the first connecting filament(s) onto or from the first handling tube. The axially movable coupling of the filament holding element to the first handling tube permits an axial relative movement of the filament holding element with respect to the first handling tube and thus an axial evasive movement of the filament holding element during radial expansion or contraction of the wire structure. In alternative embodiments, the filament holding element may be rigidly arranged in an axial direction on the first handling tube, which may be sufficient for some applications. Depending on the embodiment, the filament holding element can be an element formed on the first handling tube itself, e.g., molded on, or an element furnished separately from the latter, which is fixed to the first handling tube in a detachable or non-detachable manner.

In a further embodiment of the invention, the tube connection interface of the first handling tube has a filament holding sleeve disposed on the first handling tube in a non-rotatable manner. This represents a structurally and functionally advantageous implementation of the tube connection interface for holding the first connecting filament(s) to the first handling tube. The filament holding sleeve can, for example, be prefabricated separately and slid onto the first handling tube and fixed to it so that it cannot rotate, either detachably or non-detachably, depending on the requirements. In some embodiments, the filament holding sleeve can simultaneously form the above-mentioned filament holding element, for which purpose it is then axially movable instead of axially rigidly fixed to the first handling tube in a rotationally fixed manner.

In a further embodiment of the invention, the second connecting unit includes a second filament connecting unit comprising at least one second connecting filament which is fixed, on the one hand, to a second structural connection interface of the wire structure axially spaced from the first structural connection interface and, on the other hand, to a tube connection interface of the second handling tube and extends therebetween with a radial directional component, and which winds onto or unwinds from the second handling tube upon relative rotation of the first and second handling tubes in accordance with the direction of rotation.

In this further embodiment, the second connecting unit thus corresponds to the first connecting unit in terms of its configuration. This has the very advantageous consequence that the wire structure can be held at the first structural connection interface and at the second structural connection interface axially spaced therefrom by the respective filament connecting unit with one or more connecting filaments each on the first handling tube on the one hand and on the second handling tube on the other hand, and can be retracted radially inwards from its self-expanded, expanded configuration by winding the first connecting filament or filaments and the second connecting filament or filaments onto the first and the second handling tube, respectively, at these two axially spaced structural connection interfaces. This supports a relatively uniform radial contraction of the wire structure over its axial extension, for example in order to detach it from radially surrounding material after a misplacement and then reposition it by axial displacement.

In one embodiment of the invention, the second filament connecting unit has a plurality of second connecting filaments, each of which is fixed on the one hand to the second structural connection interface of the wire structure and on the other hand to the tube connection interface of the second handling tube and extends therebetween with a radial directional component, and which winds onto or unwinds from the second handling tube upon relative rotation of the first and second handling tubes in accordance with the direction of rotation.

This measure favors a uniform connection of the wire structure via its second structural connecting point to the second handling tube or its tube connection interface via the plurality of second connecting filaments in this case, with analogous effects and advantages as indicated above for the embodiment of the first filament connecting unit by means of the plurality of first connecting filaments. Likewise, by analogy, the wire structure at its second structural connection interface and/or the second handling tube may optionally have a number of connecting points corresponding to the number of second connecting filaments, to each of which one of the second connecting filaments may be attached. This promotes uniform application of the radial retraction force provided by the second filament connecting unit to the wire structure at multiple, preferably circumferentially interspaced points during winding of the second connecting filaments thereof onto the second structural connection interface. In alternative embodiments, the second filament connecting unit comprises only a single connecting filament, which may be sufficient for certain applications. For the second connecting filament(s), again any material suitable for their required function can be used, as indicated above for the first connecting filaments.

In a further embodiment of the invention, the second structural connection interface has a plurality of second filament attachment points distributed in a circumferential direction of the wire structure and to which the second connecting filaments are attached. Also in this case, as above with respect to the analogous embodiment of the first structural connection interface, one or more second connecting filaments may be defined at each second filament attachment point as required and appropriate for the application.

This provides, in an analogously advantageous manner as in the corresponding case of the first filament connecting unit, a circumferentially distributed and thus uniform connection of the wire structure to the second handling tube and a correspondingly circumferentially relatively uniform action of the second filament connecting unit for radially contracting the wire structure in the region of its second structural connection interface. Like the above-mentioned first connecting filaments, the second connecting filaments can be arranged in particular at equal distances from one another in the circumferential direction or, depending on requirements and the application, e.g., depending on the specific shape of the wire structure, in particular in the region of its second structural connection interface, alternatively in a distribution with different distances in the circumferential direction. In alternative implementations, the plurality of second connecting filaments are fixed at a common filament attachment point of the second structure connection interface of the wire structure, while they may be circumferentially distributed, for example, at the tube connection interface of the second handling tube.

In one embodiment of the invention, the tube connection interface of the second handling tube has a second filament holding element, which is arranged on the second handling tube in a rotationally fixed and axially movable manner and on which the at least one second connecting filament is secured, or a second filament holding sleeve, which is arranged on the second handling tube in a rotationally fixed manner and on which the at least one second connecting filament is secured. This presents advantageous embodiments for defining the second connecting filament(s) at the tube connection interface of the second handling tube with the analogous features and advantages as indicated above to some embodiments for defining the at least one first connecting filament at the tube connection interface of the first handling tube. Here, too, the second filament holding sleeve can optionally act as the second filament holding element at the same time if it is arranged such as to be axially movable instead of axially rigid on the second handling tube.

In a further embodiment of the invention, the control interface is set up for counter-rotational motion actuation of the first and second handling tubes at pre-definable rotational speeds and/or filament winding rates.

This measure can contribute to the optimization of the radial contraction movement of the wire structure by the corresponding relative rotation, initiated at the control interface, of the first and the second handling tubes for winding at least the first connecting filament and optionally also the second connecting filament.

By rotating the two handling tubes in opposite directions, e.g., one tube clockwise and the other tube counterclockwise, an overall rotation of the wire structure can be avoided or at least kept low. By suitable presetting or selection of the rotation speeds and/or filament winding rates for the handling tubes rotated relative to one another, the radial drawing-in speed during radial drawing-in of the wire structure can be set as desired, and it can be ensured, if necessary, that the wire structure is drawn in radially in the same way, e.g., at the same or approximately the same radial drawing-in speed, at the various axially spaced structure connection interfaces, even if the handling tubes have different winding diameters for the first or second connecting filament(s) in their respective winding region, i.e., at or in the vicinity of the respective tube connection interface. In alternative embodiments, the counter-rotational motion actuation of the two handling tubes at the control interface is performed by the user quasi hands-free, e.g., said user twists the two handling tubes against each other without selecting a specific rotation speed or filament winding rate.

In a further embodiment of the invention, the control interface has a gearbox for actuating the first and second handling tubes in opposite rotational directions. With this embodiment of the control interface, there is the advantage of configuring a desired counter-rotational movement of the two handling tubes via the gearbox. In this case, the user only needs to specify or execute a single actuating movement, e.g., via a manually operated control element or a motorized drive, and the gearbox then ensures that this actuating movement is suitably converted into the desired counter-rotational movement of the first and second handling tubes. In alternative embodiments, the control interface does not require such a gearbox, and the user can, for example, manually ensure the defined counter-rotational movement of the two handling tubes.

In one embodiment of the invention, the holding device comprises at least a third connecting unit comprising a third filament connecting unit which includes at least a third connecting filament which is fixed, on the one hand, to a third structural connection interface of the wire structure axially spaced from the other structural connection interface or interfaces and, on the other hand, to a third structural connection interface of the wire structure axially spaced from the other tube connection interfaces of the first handling tube or of the second handling tube or of a third handling tube of the tubular assembly axially spaced apart from the other tube connection interfaces and extends therebetween with a radial directional component and which, in the event of a relative rotation of the handling tubes, winds onto or unwinds from the relevant handling tube as a function of the direction of rotation.

This advantageous embodiment allows the wire structure to be contracted by the relative rotation of handling tubes of the tubular assembly at three or more axially spaced structural connection interfaces of the wire structure, which supports particularly uniform radial contraction of the wire structure along its axial extent. For this purpose, the holding device comprises the third and optionally one or more further connecting units with respective filament connecting unit, each formed with one or more associated connecting filaments. The connection of the third and optionally further structural connection interfaces of the wire structure by the filament connecting unit in question is made to the first handling tube or to the second handling tube or to a third and, optionally, a fourth, etc., handling tube of the tubular assembly, depending on requirements and the application. The further handling tube(s), e.g., a third, fourth, etc. handling tube, as well as the first and second handling tubes are coaxial or transversely offset components of the respective tubular assembly, which are located one inside the other and/or side by side and are movable in relative rotation with respect to one another. In alternative embodiments, the holding device comprises only the first filament connecting unit and optionally the second filament connecting unit.

Advantageous embodiments of the invention are shown in the drawings. These and other embodiments of the invention are described in more detail below. The figures show the following:

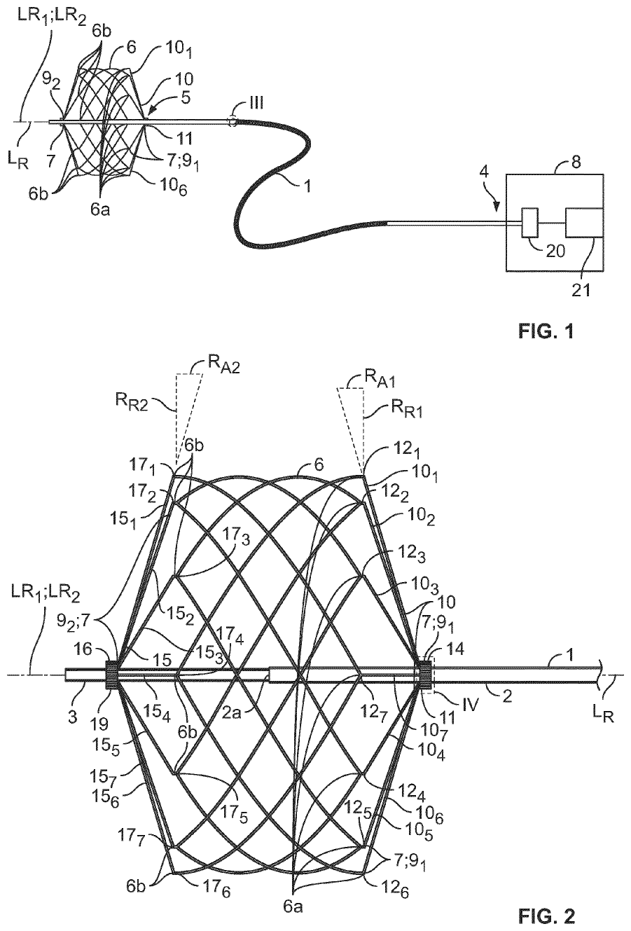

FIG. 1 is a schematic side view of a tubular instrument,

FIG. 2 is a schematic side view of a distal functional area of the tubular instrument of FIG. 1 with a radially self-expanding wire structure held thereto in an expanded state,

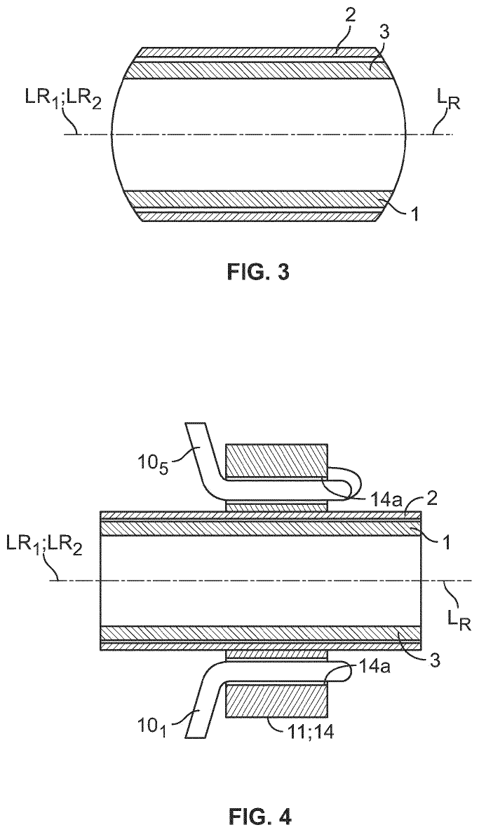

FIG. 3 is a sectional longitudinal view of a tubular assembly of the tubular instrument of FIG. 1 in a region III of FIG. 1,

FIG. 4 is a sectional longitudinal view of a region IV of FIG. 2 including a tube connection interface of a first retaining tube of the tubular assembly,

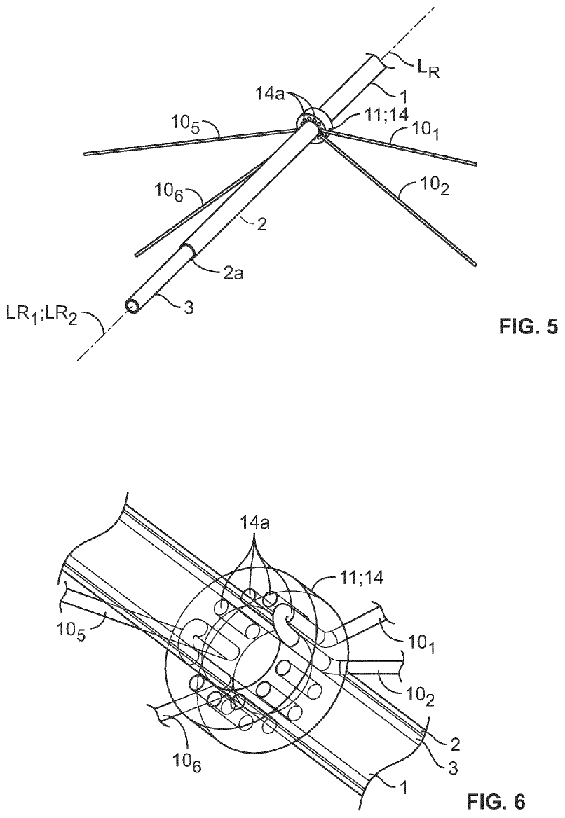

FIG. 5 is a sectional oblique frontal view of an area of the tubular assembly with the tube connection interface of the first handling tube,

FIG. 6 is a sectional oblique rear view to the tube connection interface of the first handling tube,

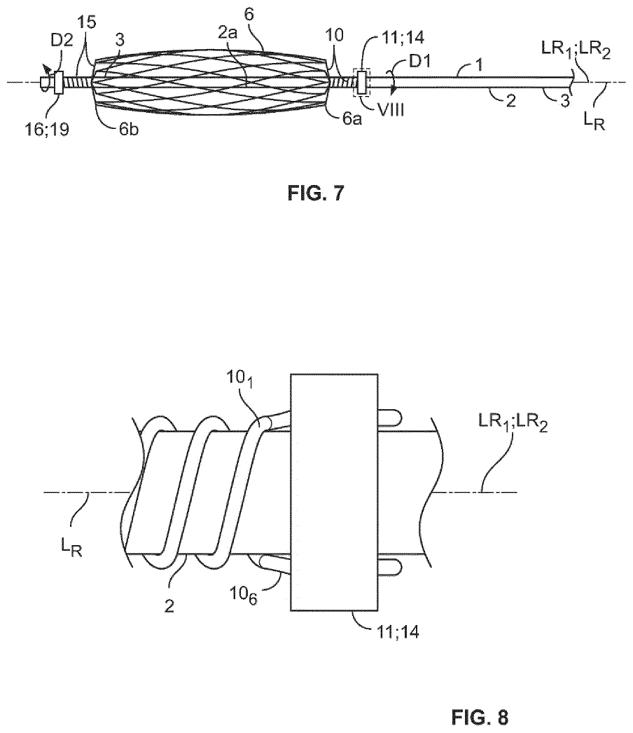

FIG. 7 is the side view of FIG. 2 with the wire structure in a radially retracted state,

FIG. 8 is a detailed view of a region VIII in FIG. 7,

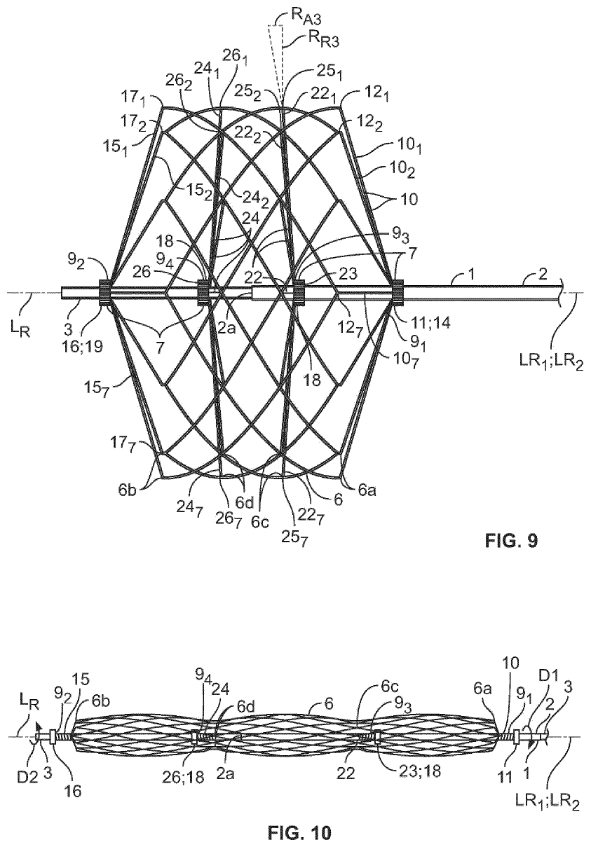

FIG. 9 is a side view according to FIG. 2 for a variant of the tubular instrument with more than two structural connection interfaces of the wire structure, and

FIG. 10 is the view of FIG. 7 for the tubular instrument variant of FIG. 9.

The tubular instrument shown in the figures in various embodiments and views includes a tubular assembly 1 comprising a first handling tube 2 and a second handling tube 3, a radially self-expanding wire structure 6, and a holding device 7. The two handling tubes 2, 3 extend from a proximal operating area 4 to a distal functional area 5, as schematically shown in FIG. 1.

In the examples shown, the handling tubes 2, 3 are arranged coaxially, i.e., their longitudinal axes LR₁ and LR₂ respectively coincide to form a common longitudinal axis L_{R} of the tubular assembly 1. In alternative embodiments, they are arranged with parallel offset longitudinal axes LR₁, LR₂ nested or adjacent to each other.

The tubular assembly 1 preferably has a desired flexibility, i.e., bendability, for which purpose its handling tubes, such as the first and second handling tubes 2, 3 in the examples shown, are made of a corresponding flexible tube material capable of bending. The tubular instrument may be, for example, a medical catheter instrument, such as one for minimally invasive implantation of artificial heart valves or vascular stents, in which case the handling tubes 2, 3 may be made of any flexible tube material known per se to the person skilled in the art for this application. In alternative embodiments, this can be a tubular instrument usable in pipeline or piping applications.

The first and second handling tubes 2, 3 of the tubular assembly 1 are arranged to be relatively rotatable with respect to each other. In particular, in the examples shown, they can be changed in their relative rotational position with respect to the common longitudinal axis of the tube L_{R}. At the proximal operating area 4, the tubular instrument has a control interface 8 for relative rotational motion actuation of the first and second handling tubes 2, 3. The control interface 8 can be of any configuration known to the person skilled in the art for carrying out the operating functions required and explained herein and is therefore only shown schematically in FIG. 1. The two handling tubes 2, 3 and the control interface 8 are configured, as required, so that only the first or only the second or both handling tubes 2, 3 is/are actively rotated, preferably in opposite directions, around the respective longitudinal axis LR₁, LR₂ or the common longitudinal axis L_{R} in order to achieve the desired relative rotation.

In the exemplary embodiments shown, the wire structure 6 has a cylindrical, wire mesh-like shape, such as is suitable for applications in artificial heart valves and vascular stents. It goes without saying that for other applications, the wire structure (6) may have a different shape adapted to its intended use.

Depending on the application, the wire material used to build the wire structure 6 can be, for example, a shape memory metallic alloy material or another elastic metal or plastic material. The wire structure 6 can be cut in one piece from a tube material by means of laser cutting, as is known for stent structures per se, for example, or alternatively be assembled from several prefabricated individual parts.

The holding device 7 is configured to hold the radially self-expanding wire structure 6 on the distal functional area 5 of the tubular assembly 1. To this end, the holding device 7 comprises a first connecting unit 9₁ connecting the wire structure 6 to the first handling tube 2, and a second connecting unit 9₂ connecting the wire structure 6 to the second handling tube 3.

The first connecting unit 9₁ contains a first filament connecting unit 10 with at least one first connecting filament 10₁, which is fixed on the one hand at a first structural connection interface 6a of the wire structure 6 and on the other hand at a tube connection interface 11 of the first handling tube 2. For applications where the wire structure 6 is to remain in a positioned location of use, the at least one first connecting filament 10₁ is preferably fixed by a detachable connection at the first structure connection interface 6a of the wire structure 6. Between the first structural connection interface 6a of the wire structure 6 and the tube connection interface 11 of the first handling tube 2, the first connecting filament 10₁ extends with a radial directional component R_{R1}, wherein it optionally also extends with an axial directional component R_{A1}, as illustrated in FIG. 2, and/or a circumferential directional component.

During a relative rotation of the first and the second handling tube 2, 3 around the respective longitudinal axis LR₁, LR₂ or the common longitudinal axis L_{R} respectively, the first connecting filament 10₁ winds onto or off the first handling tube 2 depending on the direction in which the first handling tube 2 rotates relative to the second handling tube 3, i.e. clockwise or counterclockwise.

Since the at least one first connecting filament 10₁ extends between the wire structure 6 and the first handling tube 2 with a said radial directional component R_{R1}, its winding onto the first handling tube 2 has the effect of shortening it radially in the process, as a result of which it pulls the wire structure 6 radially inwards at the relevant first structural connection interface 6a.

This makes it possible for the user to radially retract the wire structure 6 from an expanded state, e.g. according to FIGS. 2 and 9, into the retracted state, e.g. according to FIGS. 7 and 10, respectively, by relative rotation of the handling tubes 2, 3 in the corresponding direction of rotation. In this case, the state according to FIGS. 2 and 9, respectively, need not necessarily be a fully expanded state of the wire structure 6, and the state according to FIGS. 7 and 10, respectively, need not necessarily be a fully retracted state thereof. In this respect, the wire structure 6 can be retracted this way, by an operating action of the user at the proximal end region 4 of the tubular instrument, from a completely or partially expanded state, into which it automatically expands on account of its radially self-expanding characteristic, for example until it comes to bear against radially surrounding material, into a state which, in contrast, is radially retracted to a certain extent.

This allows the user to easily reposition the wire structure 6 remotely in the event that it has expanded in an incorrect position. Once the radially retracted wire structure 6 has been repositioned, for example by axially displacing and/or rotating the tubular assembly 1 and thus also the wire structure 6, the user can enable the automatic radial expansion of the wire structure again by performing a relative rotation of the two handling tubes 2, 3 in the opposite direction to the previous one, as a result of which the at least one connecting filament 10₁ unwinds from the first handling tube 2 again and releases the radial expansion of the wire structure 6. Once the desired exact position of the wire structure 6 has been achieved, its connection to the tubular assembly 1 may be released, after which the tubular assembly 1 may be withdrawn from the location of use of the wire structure. Alternatively, if the wire structure 6 serves merely as a transport medium or carrier for depositing another object or substance at the relevant location of use, it may again be retracted radially by relative rotation of the handling tubes 2, 3 and then withdrawn from the location of use together with the tubular assembly 1.

In advantageous embodiments, as in the examples shown, the first filament connecting unit 10 includes a plurality of first connecting filaments 10₁, 10₂, etc., each of which is fixed, on the one hand, to the first structural connection interface 6a of the wire structure 6 and, on the other hand, to the tube connection interface 11 of the first handling tube 2 and extends therebetween with a radial directional component, wherein the radial directional components for the different connecting filaments 10₁, 10₂, etc., may be the same size or alternatively different. Each of the first connecting filaments 10₁, 10₂, etc., winds onto or unwinds from the first handling tube 2 during said relative rotation of the first and second handling tubes 2, 3 depending on the rotational direction of said relative rotation, i.e., during a certain relative rotation of the two handling tubes 2, 3 all first connecting filaments 10₁, 10₂, etc, are either wound onto the first handling tube 2 or unwound therefrom. The presence of the plurality of first connecting filaments 10₁, 10₂, etc. may be conducive to a uniform radial retraction of the wire structure 6 around the circumference thereof.

In some embodiments, as in the examples shown, the first structural connection interface 6a of the wire structure 6 includes a plurality of first filament attachment points 12₁, 12₂, etc., arranged in a circumferential direction of the wire structure 6 in a distributed manner and to which the first connecting filaments 10₁, 10₂, etc., are attached. This measure is conducive to a uniform radial retraction of the wire structure 6 along its circumference, in that the retraction force acts via the first connecting filaments 10₁, 10₂, etc., on the filament attachment points 12₁, 12₂, etc., distributed along the circumference of the wire structure 6. In this case, the number of first filament attachment points 12₁, 12₂, etc., may be equal to or less than the number of first connecting filaments 10₁, 10₂, etc. In the latter case, at least two first connecting filaments are fixed together at a same first filaments attachment point.

In some embodiments, as in the examples shown, the first and second handling tubes 2, 3 are arranged to move relative to each other in the axial direction, i.e. parallel to the longitudinal tube axis L_{R}. This allows the tubular assembly 1 to yield to or accommodate an axial change in length of the wire structure 6 in appropriate embodiments.

This can be seen, for example, from a comparative view of FIGS. 2 and 7 in that, starting from the expanded state of the wire structure 6 according to FIG. 2, when the wire structure 6 is radially contracted into the retracted state according to FIG. 7, the second handling tube 3 moves axially out of the first handling tube 3 to a certain extent.

In some embodiments of the tubular instrument, the tubular connection interface 11 of the first handling tube 2 has, as in the examples shown, a filament holding sleeve 14 arranged non-rotatably on the first handling tube 2. That is, when the first handling tube 2 is rotated, the filament holding sleeve 14 rotates therewith. The at least one first connecting filament 10₁ is fixed onto the filament holding sleeve 14. Specifically, in the examples shown, the plurality of first connecting filaments 10₁, 10₂, etc., are held to the filament holding sleeve 14. In the examples shown, this is realized in that the filament holding sleeve 14 is provided with a plurality of circumferentially distributed axial bores 14a, as can be seen in FIGS. 4 and 5 and in FIG. 6 reproduced in a partially transparent representation for this purpose, and a respective filament piece coming from the first structural connection interface 6a of the wire structure 6 is looped through one of the bores 14a in the proximal direction and looped back again while being deflected through an adjacent bore 14a in the distal direction and returned to the first structural connection interface 6a of the wire structure 6. This type of connecting filament attachment to the tube connection interface 11 of the first handling tube 2 is shown in FIGS. 4 to 6 as an example for two pieces of filament for providing the connecting filaments 10₁ and 10₂ or 10₅ and 10₆, respectively. Consequently, in this case, at least two of the first connecting filaments 10₁, 10₂, etc., are formed by a single piece of filament held on the filament holding sleeve 14 by the deflected double looping through. As required, the piece of filament is fixed at the first structural connection interface 6a of the wire structure 6 or at its filament attachment points 12₁. 12₂, etc., with a respective filament end, e.g., by means of a detachable or non-detachable knot, or also looped through there in a deflecting manner and continued. The filament holding sleeve 14, for example, together with the first connecting filaments 10₁, 10₂, etc., held thereon, can be slid onto or otherwise positioned against the first handling tube 2 and subsequently fixed thereto in a releasable or non-releasable manner.

Alternatively, the attachment of the filament holding sleeve 14 to the first handling tube 2 may take place before the attachment of the first connecting filament 10₁, 10₂, etc., to the filament holding sleeve 14.

In some embodiments, as in the examples shown, the second connecting unit 9₂ comprises a second filament connecting unit 15 having at least one second connecting filament 15₁ fixed, on the one hand, to a second structural connection interface 6b of the wire structure 6 axially spaced from the first structural connection interface 6a and, on the other hand, to a tube connection interface 16 of the second handling tube 3. Analogous to the case of the at least one first connecting filament 10₁, the at least one second connecting filament 15₁ again extends with a radial directional component R_{R2} and optionally with an axial directional component R_{A2} and/or a circumferential directional component, as illustrated in FIG. 2, and winds onto or off the second handling tube 3 upon relative rotation of the first and second handling tubes 2, 3 depending on the direction of rotation.

In the examples shown, to prevent this winding and unwinding of the at least one second connecting filament 15₁ onto and from the second handling tube 3 from being obstructed by the surrounding first handling tube 2, the second handling tube 3 extends distally forward beyond a distal end 2a of the first handling tube 2, wherein the second structural connection interface 6b of the wire structure 6 lies distally in front of the first structural connection interface 6a at an axial distance, and the tube connection interface 16 of the second handling tube 3 is arranged in the region of the second handling tube 3 lying distally in front of the distal end 2a of the first handling tube 2.

Consequently, in this configuration, the second connecting unit 9₂ corresponds in its function to that of the first connecting unit 9i. The wire structure 6 is radially retracted at its second structural connection interface 6b by winding the at least one second connecting filament 15₁ onto the second handling tube 3. Unwinding the second connecting filament 15₁ from the second handling tube 3 releases the self-expanding function for the wire structure 6 at its second structural connection interface 6b.

The optional axial directional component R_{A1}, R_{A2} of the connecting filaments 10₁, 10₂, etc., 15₁, 15₂, etc., can influence how the connecting filaments 10₁, 10₂, etc., 15₁, 15₂, etc., with their successive windings wind onto the respective handling tube 2, 3. In the examples shown, the respective connecting filament 10₁, 10₂, etc., 15₁, 15₂, etc., is wound helically in such a way that its windings follow one another on the handling tube 2, 3 in a single layer with axial spacing, or alternatively without axial spacing, as is schematically illustrated in FIGS. 7 and 10 and in FIG. 8 for the two connecting filaments 10₁ and 10₆ in more detail. Alternatively, the connecting filament windings on the relevant handling tube 2, 3 may be radially superimposed, e.g., if the relevant connecting filament extends without the axial directional component R_{A1}, R_{A2} or with a correspondingly small axial directional component R_{A1}, R_{A2}.

In some embodiments, as in the examples shown, the second filament connecting unit 15 has a plurality of second connecting filaments 15₁, 15₂, etc., each of which is fixed on the one hand to the second structural connection interface 6b of the wire structure 6 and on the other hand to the tube connection interface 16 of the second handling tube 3 and extends therebetween with said radial directional component and winds onto or unwinds from the second handling tube 3 in accordance with the direction of rotation upon relative rotation of the two handling tubes 2, 3. In this case, the second filament connecting unit 15 is designed analogously to the design of the first filament connecting unit 10 with the plurality of first connecting filaments 10₁, 10₂, etc., and accordingly has analogous properties and functions, including the advantages mentioned in this regard above with respect to uniform radial retracting of the wire structure 6 over its circumference.

In some implementations, the second structural connection interface 6b of the wire structure 6 has, as in the examples shown, a plurality of second filament attachment points 17₁, 17₂, etc., distributed in a circumferential direction of the wire structure 6 and to which the second connecting filaments 15₁, 15₂, etc., are fixed. As required, the second connecting filaments 15₁, 15₂, etc., are also fixed at the second filament attachment points 17₁, 17₂, etc., detachably or non-detachably.

In this embodiment, the second structural connection interface 6b corresponds in its properties and functions to the first structural connection interface 6a of the wire structure 6 in its embodiment with the plurality of first filament attachment points 12₁, 12₂, etc. This also applies in particular with regard to the advantages mentioned for this purpose above with respect to uniform radial retraction of the wire structure 6 over its circumference.

In some embodiments, as in the examples shown, the tube connection interface 16 of the second handling tube 3 comprises a second filament holding sleeve 19 arranged non-rotatably on the second handling tube 3, on which the at least one second connecting filament 15₁ is retained. In this embodiment, the tube connection interface 16 of the second handling tube 3 consequently corresponds in its functions and properties to the tube connection interface 11 of the first handling tube 2 explained above in the embodiment with the filament holding sleeve. This also applies to the optional possibility that the second filament holding sleeve 19 is provided with a plurality of axial bores, through which one or more filament pieces can be looped with deflection, in order to provide the second connecting filaments 15₁, 15₂, etc., as explained above for the analogous filaments looping through at the first filaments holding sleeve 14. And analogous to the first filament holding sleeve 14, the second filament holding sleeve 19, for example, together with the second connecting filaments 15₁, 15₂, etc., held thereon, can be slid onto or otherwise placed against the second handling tube 3 and subsequently fixed thereto in a detachable or non-detachable manner. Alternatively, the attachment of the second filament holding sleeve 19 to the second handling tube 3 can take place prior to the attachment of the second connecting filaments 15₁, 15₂, etc., to the second filament holding sleeve 19.

In some embodiments of the tubular instrument, the control interface 8 is set up for counter-rotational motion actuation of the first and second handling tubes 2, 3 at pre-definable rotational speeds and/or filament winding rates. For this purpose, the control interface 8 can, for example, be equipped with a suitable operating control 21, as illustrated in FIG. 1 for the embodiment of the tubular instrument there. The operating control 21 is optionally designed as an automated control system comprising, among other things, a motorized drive, or as a manual control system.

In some embodiments of the tubular instrument, the control interface comprises a gearbox 20 for counter-rotational motion actuation of the first and second handling tubes 2, 3, as also shown in FIG. 1 for the instrument embodiment there. By means of an appropriate design of the gearbox 20, a respective desired type of relative rotation of the two handling tubes 2, 3 can be specified or set, e.g. active rotation of both tubes 2, 3 in opposite rotation direction with the same or different rotational speeds and/or filament winding rates. This makes it possible to specify a desired time sequence for the radial retraction movement of the wire structure 6.

In some embodiments, as in the embodiment example of FIGS. 9 and 10, the holding device 7 comprises at least one third connecting unit 9₃ having a third filament connecting unit 22 which includes at least one third connecting filament 22₁ that is fixed, on the one hand, to a third structural connecting interface 6c of the wire structure 6 axially spaced from the other structural connecting interface(s) 6a, 6b and, on the other hand, to a further tubular connecting interface 23 axially spaced from the other tube connection interfaces 11, 16 and extending therebetween with a radial directional component R_{R3} and optionally an axial directional component R_{A3}. In this case, the further tube connection interface 23 can be located on the first handling tube 2 or on the second handling tube 3 or on a further, third handling tube of the tubular assembly 1, depending on the application, whereby it winds onto or unwinds from the handling tube in question depending on the rotational direction during a relative rotation of the handling tubes involved.

In the embodiment example of FIGS. 9 and 10, in addition to the third connecting unit 9₃ comprising the third filament connecting unit 22, a fourth connecting unit 9₄ comprising a fourth filament connecting unit 24 including at least a fourth connecting filament 24₁ is provided, wherein both further filament connecting units 22, 24 in this example each include a plurality of third connecting filaments 22₁, 22₂, etc., and fourth connecting filaments 24₁, 24₂, etc., respectively. The third connecting filaments 22₁, 22₂, etc., are fixed to the third structural connection interface 6c of the wire structure 6, again at a plurality of associated third filament attachment points 25₁, 25₂, etc., and the fourth connecting filaments 24₁, 24₂, etc., are fixed to a fourth structural connection interface 6d of the wire structure 6, again at a plurality of associated filament attachment points 26₁, 26₂, etc., as explained for the first and second connecting units 9₁, 9₂ and their filament connecting units 10, 15.

In other words, the four connecting units 9₁ to 9₄ may be of the same design among themselves, but in alternative embodiments they may be of different designs. In the embodiment example of FIGS. 9 and 10, the tube connection interface 23, at which the third connecting filaments 22₁, 22₂ are fixed, is located at the first handling tube 2, and the fourth connecting filaments 24₁, 24₂, etc., are fixed at another tube connection interface 26, which is located at the second handling tube 3.

In the embodiment example of FIGS. 9 and 10, the two additional tube connection interfaces 23, 26 are each formed by a further filament holding sleeve in the same way as the two other tube connection interfaces 11, 16. If an axial evasive movement is desired between the tube connection interfaces 11 and 23 or 16 and 26 belonging to the same handling tube 2 or 3 in each case, in order to absorb an axial change in length of the wire structure 6 during radial retraction, at least one of these respective two tube connection interfaces 11, 23 or 16, 26 may be designed (as an alternative or in addition to the embodiment as a filament holding sleeve) as a filament holding element 18 which is arranged on the associated handling tube 2, 3 in a rotationally fixed and axially movable manner and to which the connecting filaments concerned are attached, as is indicated in the example in FIGS. 9 and 10 as an example for the two additional tube connection interfaces 23, 26. The axially movable connection of the respective filament holding element 18 to the associated handling tube 2, 3 can be realized in any conventional manner, e.g., by an axial slotted guide system with an axial groove on one part and a corresponding link cam engaging therein on the other part.

In other embodiments, no axially movable connection of the tube connection interfaces 11, 16, 23, 26 to the respective handling tube 2, 3 and/or no axial movability of the handling tubes 2, 3 relative to one another is provided, which is possible in particular for applications in which the wire structure does not change in its axial length during radial retraction in the region of its structure connection interfaces or, in any case, does not require such an axial length change.

FIGS. 7 and 10 illustrate, for the two examples in question, the winding motion for radially retracting the wire structure 6 from its expanded state as displayed in FIGS. 2 and 9, respectively, by actively rotating the first handling tube 2 counterclockwise as symbolized by a rotary arrow D1 and actively rotating the second handling tube 3 clockwise as symbolized by a rotary arrow D2. As a result of this counterclockwise, active relative rotation of the two handling tubes 2, 3 with respect to each other, the first connecting filament(s) 10₁, 10₂, etc., wind onto the first handling tube 2, and the second connecting filament(s) 15₁, 15₂, etc., wind onto the second handling tube 3, causing the wire structure 6 to be radially contracted via its two axially spaced structural connection interfaces 6a, 6b. In the example of FIGS. 9 and 10, the third connecting filament(s) 22₁, 22₂, etc., additionally wind onto the first handling tube 2, and the fourth connecting filament(s) 24₁, 24₂, etc., wind onto the second handling tube 3.

Preferably, the same rotational amplitudes or rotational speeds or the same filament winding rates are selected for the active rotation of the two handling tubes 2, 3 in opposite directions, i.e. the first handling tube 2 is rotated by the same angle in one direction as the second handling tube 3 in the opposite direction. As a result, the wire structure 6 as a whole remains essentially stationary in its rotational position, i.e. it is drawn in radially without rotating in a noticeable manner. This is particularly convenient for many applications, e.g., it can minimize any tissue damage when the wire structure 6 in an application for an artificial heart valve or vascular stent is to be detached from the surrounding tissue after misplacement for repositioning. In addition, it is generally advantageous if the handling tubes 2, 3 are rotated in such a way that all connecting filaments wind onto the respective handling tube 2, 3 with essentially the same filament length per unit of time. This allows the wire structure 6 to be radially retracted at virtually the same radial retraction speed at all of its structural connection interfaces, and thus relatively uniformly over its axial extent.

As the embodiments shown and the other embodiments explained above make clear, the invention provides, in a highly advantageous manner, a tubular instrument having a self-expanding wire structure that permits repositioning of the wire structure, if necessary, when it is in an expanded, misplaced state, without damaging surrounding material to which it has bonded by the independent radial expansion. The tubular instrument according to the invention is particularly suitable for medical applications in endoscopy technology, e.g. as a medical catheter instrument for minimally invasive implantation of prosthetic heart valve and vascular stents, but can also be used for non-medical applications in suitable embodiments.

1. A tubular instrument, in particular a medical catheter instrument, comprising:
a tubular assembly comprising first and second handling tubes extending from a proximal operating area to a distal functional area;
a radially self-expanding wire structure; and
a holding device adapted to hold the radially self-expanding wire structure at the distal functional area of the tubular assembly,
wherein the first and second handling tubes are arranged to be relatively rotatable with respect to each other,
a control interface is provided at the proximal operating area for the relative rotational motion actuation of the first and second handling tubes, and
the holding device comprises a first connecting unit connecting the wire structure to the first handling tube, and a second connecting unit connecting the wire structure to the second handling tube,
wherein the first connecting unit includes a first filament connecting unit having at least one first connecting filament which is fixed, on the one hand, to a first structural connection interface of the wire structure and, on the other hand, to a tube connection interface of the first handling tube and extends therebetween with a radial directional component and which, upon a relative rotation of the first and second handling tubes, winds onto or unwinds from the first handling tube in dependence on the direction of rotation.

2. The medical catheter instrument according to claim 1, wherein the first filament connecting unit comprises a plurality of first connecting filaments, each of which is fixed on the one hand to the first structure connection interface of the wire structure and on the other hand to the tube connection interface of the first handling tube and extends therebetween with a radial directional component and winds onto or unwinds from the first handling tube upon relative rotation of the first and second handling tubes in accordance with the direction of rotation.

3. The medical catheter instrument according to claim 2, wherein the first structural connection interface comprises a plurality of first filament attachment points distributed in a circumferential direction of the wire structure and to which the first connecting filaments are attached.

4. The medical catheter instrument according to claim 1, wherein the first and second handling tubes are arranged for relative movement with respect to each other in the axial direction parallel to the longitudinal axis of the tube.

5. The medical catheter instrument according to claim 1, wherein the tube connection interface of the first handling tube comprises a filament holding element which is arranged on the first handling tube in a rotationally fixed and axially movable manner and on which the at least one first connecting filament is fixed.

6. The medical catheter instrument according to claim 1, wherein the tube connection interface of the first handling tube comprises a filament holding sleeve arranged non-rotatably on the first handling tube, on which the at least one first connecting filament is fixed.

7. The medical catheter instrument according to claim 1, wherein the second connecting unit includes a second filament connecting unit having at least one second connecting filament fixed, on the one hand, to a second structural connection interface of the wire structure axially spaced from the first structural connection interface and, on the other hand, to a tube connection interface of the second handling tube and extending therebetween with a radial directional component and which, upon a relative rotation of the first and second handling tubes, winds onto or unwinds from the second handling tube in dependence on the direction of rotation.

8. The medical catheter instrument according to claim 7, wherein the second filament connecting unit comprises a plurality of second connecting filaments, each of which is fixed on the one hand to the second structural connection interface of the wire structure and on the other hand to the tube connection interface of the second handling tube and extends therebetween with a radial directional component and winds onto or unwinds from the second handling tube upon relative rotation of the first and second handling tubes in accordance with the direction of rotation.

9. The medical catheter instrument according to claim 8, wherein the second structural connection interface comprises a plurality of second filament attachment points distributed in a circumferential direction of the wire structure and to which the second connecting filaments are attached.

10. The medical catheter instrument according to claim 6, wherein the tube connection interface of the second handling tube comprises a second filament holding element which is arranged on the second handling tube in a rotationally fixed and axially movable manner and on which the at least one second connecting filament is held.

11. The medical catheter instrument according to claim 6, wherein the tube connection interface of the second handling tube comprises a second filament holding sleeve which is arranged on the second handling tube in a rotationally fixed manner and on which the at least one second connecting filament is held.

12. The medical catheter instrument according to claim 1, wherein the control interface is set up for counter-rotational motion actuation of the first and second handling tubes at pre-definable rotational speeds or filament winding rates.

13. The medical catheter instrument according to claim 1, wherein the control interface comprises a gearbox for actuating the first and second handling tubes in opposite rotational directions.

14. The medical catheter instrument according to claim 7, wherein the holding device comprises at least a third connecting unit comprising a third filament connecting unit which includes at least a third connecting filament which is fixed, on the one hand, to a third structural connection interface of the wire structure axially spaced from the other structural connection interface or interfaces and, on the other hand, to a further structural connection interface of the wire structure axially spaced from the other tube connection interfaces of the first handling tube or of the second handling tube or of a third handling tube of the tubular assembly axially spaced apart from the other tube connection interfaces and extends therebetween with a radial directional component and which, in the event of a relative rotation of the handling tubes, winds onto or unwinds from the relevant handling tube as a function of the direction of rotation.
